(19) United States
(12) Patent Application Publication
(10) Pub. No.: US 2007/0149840 A1
(43) Pub. Date: Jun. 28, 2007

(54) Technology to improve yield and capacity of the butadiene extraction facility which uses C4 as feedstock supplied by ethylene plant

(75) Inventors: Minghua Wang, Beijing (CN); Duoshan Zhao, Beijing (CN); Meng Wang, Beijing (CN)

Correspondence Address: Jingcheng Wu, 3rd Floor, 253 West 35th Street, New York, NY, 10001, US

(73) Assignee: Minghua Wang, Beijing (CN)

(21) Appl. No.: 11/642,265

(22) Filed: Dec. 20, 2006

(30) Foreign Application Priority Data
Dec. 26, 2005 (CN) 200510132525.1

(51) Int. Cl. C07C 7/00

(52) U.S. Cl. 585/809

(57) ABSTRACT

This technology aims to improve yield and throughput of butadiene extraction facility which uses C₄ as feedstock supplied by ethylene plant. The key point of the technical solution is to properly broaden quality index of trans-butene-2 and cis-butene-2 in secondary rectification tower. In addition, an alkyne rectification tower is added to the extraction process. The main application is to improve the yield of butadiene extraction facility and its throughput. The major benefits are that butadiene yield is improved by 1% to 2% and the throughput is increased by 10%.

A technology to improve yield and capacity of the butadiene extraction facility which uses C₄ as feedstock supplied by ethylene plant

### CROSS-REFERENCE TO RELATED APPLICATIONS

(Not Applicable)

### STATEMENT REGARDING FEDERALLY SPONSORED RESEARCH OR DEVELOPMENT

(Not Applicable)

### REFERENCE TO A SEQUENCE LISTING, A TABLE, OR A COMPUTER PROGRAM LISTING COMPACT DISC APPENDIX

(Not Applicable)

### BACKGROUND OF THE INVENTION

Based on different solvents, there are three approaches to butadiene extraction: acetonitrile extraction method (ACN), dimethylformamide method (DMF), and N-methyl-2-pyrrolidinone (NMP) extraction technology.

The ACN method produces butadiene yield 96% to 97%, while the DMF and NMP process enable yields above 97%. With the rapid development of the auto industry in China, the demand for tires and other rubber products has been increasing, and so does it for butadiene—the main material for rubber production. As a consequence, many butadiene extraction facilities are running at full capacity or even overloaded. More and more facilities are added into operation to fulfill the production needs, and the scale is getting bigger. Some factories even have two or three butadiene extraction facilities running at the same time. To improve productivity by adding physical production facilities requires large investments and long profit cycles. However, by improving the production efficiency, the amount of investments can be reduced and the profits can be generated immediately.

### BRIEF SUMMARY OF THE INVENTION

This invention aims to improve yield and capacity of the butadiene extraction facility which uses C₄ as feedstock supplied by ethylene plant. The approach is to loosen the quality index on trans-butene-2 and cis-butene-2 in the feedstock of the secondary rectification tower and quality control index of butadiene-1.3 in the tower. In addition, an alkyne rectification tower is added into the extraction process. As a result, butadiene yield is improved by 1% to 2% and the throughput is increased by 10%.

### BRIEF DESCRIPTION OF THE SEVERAL VIEWS OF THE DRAWING

(Not Applicable)

### DETAILED DESCRIPTION OF THE INVENTION

The ACN, DMF, and NMP butadiene extraction methods enable yields at about 97%. The rest 3% is lost in the butadiene dimmer, the butane-butene fraction, the methyl acetylene off-gas, the vinyl acetylene off-gas, and the C₄C₅ fraction in the secondary rectification tower. During the design and production process, the content indexes of butadiene contained in the above mentioned gases are fixed. The amount of butadiene lost in the methyl acetylene off-gas and the vinyl acetylene off-gas is associated with the amount of methyl acetylene and vinyl acetylene in the C₄ feedstock. The higher the methyl acetylene and vinyl acetylene contents C₄ feedstock contains, the more butadiene is lost.

Because the lost content of butadiene in vinyl-acetylene off-gas accounts for great proportion in the total butadiene loss, in order to decrease the loss of butadiene, efforts have been mostly aimed at vinyl-acetylene off-gas. The approach is to increase the amount of vinyl acetylene in the off-gas in order to reduce the amount of butadiene. In the meanwhile, in order to guarantee safety, butane-butene fractions are added for dilution. This method solves the problem to a certain degree, however, a big amount of butadiene is still lost.

This invention aims to properly broaden quality index of trans-butene-2 and cis-butene-2 in the feedstock of the secondary rectification tower and 1, 3-butadiene in the tower. Broadening the quality control index of the secondary rectification tower offers an important condition to improve the throughput of butadiene extraction facility.

In addition, an alkyne rectification tower is added into the extraction process. Methyl-acetylene off-gas, vinyl-acetylene off-gas, butane-butene and waste C₄C₅ without inhibitor are fed into the alkyne rectification tower. Methyl-acetylene is eliminated from the top of the tower and vinyl-acetylene is removed from the bottom of the tower. Butadiene discharged from sidetrack will go into feedstock vaporizer. The alkyne rectification tower can reclaim a great mass of butadiene from methyl-acetylene off-gas, vinyl-acetylene off-gas and waste C₄C₅ to improve the yield of butadiene accordingly. The recovery of a great amount of butadiene from waste C₄C₅ offers the second condition for improving throughput of butadiene extraction facility.

Besides adding an alkyne rectification tower, current process conditions and pipeline connection need to be adjusted accordingly. The benefits of this invention are: 1) the yield of butadiene is improved by 1-2% and 2) the throughput is improved by around 10%.

1. This invention is a technology to improve yield and throughput of butadiene extraction facility. The feature is the improvement of butadiene extraction facility in terms of yield and throughput.

2. The feature of this invention is to properly broaden quality index of trans-butene-2 and cis-butene-2 in the feedstock of the secondary rectification tower and the quality control index of butadiene-1.3 in tower.

3. Another feature of this invention is to add an alkyne rectification tower to the butadiene extraction process. Methyl acetylene off-gas, vinyl acetylene off-gas, butane-butene and waste C₄C₅ without inhibitor are fed into alkyne rectification tower to remove methyl acetylene and vinyl acetylene, and butadiene returns to feed vaporizer.
